(19) United States
(12) Patent Application Publication
(10) Pub. No.: US 2007/0203085 A1
(43) Pub. Date: Aug. 30, 2007

(54) Methods For Interfering With Fibrosis

(76) Inventor: Florian Lang, Tuebingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/592,111

(22) PCT Filed: Feb. 8, 2005
(86) PCT No.: PCT/EP05/01246

§ 371 Date: Sep. 8, 2006

(30) Foreign Application Priority Data
Mar. 11, 2004 (EP) 04005767.1

(51) Int. Cl. A61K 48/00; A61K 31/56

(52) U.S. Cl. 514/44; 514/171

(57) ABSTRACT

Modulation of the of glucocorticoid inducible kinases to restore Connective tissue growth factor activity. Also disclosed are methods and compounds useful for the detection and treatment of fibroproliferative disorders.

### FIELD OF THE INVENTION

The current work relates to a method for altering Connective tissue growth factor (CTGF) activity comprising, contacting cells expressing serum and glucocorticoid inducible kinases SGK1 with a substance that modulates said glucocorticoid inducible kinase. Furthermore the invention relates to the diagnosis and treatment of fibrosing diseases.

### BACKGROUND OF THE INVENTION

Fibrosis is a pathological condition in which the normal wound healing process is out of balance resulting in a persistent formation of scar tissue, which hinders proper tissue functions and may lead to organ failure in a wide range of fibrosing disorders.

It is known that CTGF expressed by fibroblasts plays a key role in fibrosis and is thus an attractive target for anti-fibrotic therapies. A growing body of clinical evidence supports the role of CTGF in fibrosing disorders. Numerous published studies show that CTGF is present in abnormally high amounts in samples obtained from patients with fibroproliferative disorders of the major organs and tissues including the lungs, skin, kidneys, liver, heart, and eyes (Ihn 2002). Alterations in the generation of CTGF have been observed in a number of diseases such as irradiation, tumors, vascularization, granulomatous diseases, organ and graft rejection, lupus erythematosus, arteriosclerosis, hypoxia, oxidative stress, myocardial infarction and ischemia, cardiac hypertrophy and fibrosis, glomerulonephritis and glomerulosclerosis, renal fibrosis, diabetes mellitus, fibrosing pancreatitis, liver cirrhosis, steatohepatitis and biliary fibrosis, fibrosing and inflammatory bowel diseases, peptic ulcers, intra-abdominal adhesions, peritoneal fibrosis in peritoneal dialysis, pulmonary fibrosis, fibrosing alveolitis, pulmonary sarcoidosis and/or asthma, ovarian dysfunction, uterus myoma, arthritis, muscle pain/myalgia and fasciitis, scleroderma, keloid, gingival hypertrophy, formation of scars or connective tissue in diverse organs including in the cornea, occular fluid and in the retina, glaucoma, cerebral lesions including cerebral infarction, Alzheimer's disease, wound healing, healing after tooth extraction, bone healing and growth, bone fracture repair, herein thereafter referred to a “fibroproliferative disorders”.

Due to the central role in triggering the chain of events leading to the induction of CTGF and the following events in wound scarring process, CTGF has been suggested as a target for anti-fibrotic therapies. However such approaches are still at an early stage of research and development and the outcome is uncertain. The current application delivers a different approach, which will as well lead to interference with CTGF activity, it interfere with the regulation of CTGF at a much earlier stage thus preventing CTGF expression. Therefore the invention is expected to deliver therapeutics that have the advantage to provide significant clinical benefit without broad side effects.

SGK1 was originally cloned as glucocorticoid inducible gene and subsequently shown to be strongly up-regulated by mineral corticoids. SGK1 has been shown to be regulated through insulin like growth factor IGF1, insulin and through oxidative stress via a signal cascade involving phophoinositol-3-kinase (PI3 kinase) and phosphoinositol-dependent kinase PDK1 (Kobayashi & Cohen 1999, Park et al. 1999, Kobayashi et al. 1999). The activation of SGK1 through PDK1 involves phosphorylation of serine 422. It has furthermore been shown, that a mutation of ser 422 to aspartate (^{S422D}SGK1) results in a continuously activated kinase (Kobayashi et al. 1999).

For the measurement of glucocorticoid inducible kinase SGK1 activity various assay systems are available. In scintillation proximity assay (Sorg et al., J. of. Biomolecular Screening, 2002, 7, 11-19) and flashplate assay the radioactive phosphorylation of a protein or peptide as substrate with γATP will be measured. In the presence of an inhibitory compound no or decreased radioactive signal is detectable. Furthermore homogeneous time-resolved fluorescence resonance energy transfer (HTR-FRET), and fluorescence polarization (FP) technologies are useful for assay methods (Sills et al., J. of Biomolecular Screening, 2002, 191-214). Other non-radioactive ELISA based assay methods use specific phospho-antibodies (AB). The phospho-AB binds only the phosphorylated substrate. This binding is detectable with a second peroxidase conjugated anti sheep antibody by chemiluminescence (Ross et al., 2002, Biochem. J., immediate publication, manuscript BJ20020786).

Earlier results showed that SGK1 is a potent stimulator of the renal epithelial Na⁺-channel (De la Rosa et al. 1999, Boehmer et al. 2000, Chen et al. 1999, Naray-Fejes-Toth et al. 1999, Lang et al. 2000, Shigaev et al. 2000, Wagner et al. 2001).

Another finding related to SGK1 was that a single nucleotide polymorphism (SNP) in exon 8 with nucleotide combinations of (CC/CT) and an additional polymorphism in intron 6 (CC) are associated with increased blood pressure (Busjahn et al. 2002) and from this it was concluded that SGK1 may be important to blood pressure regulation and hypertension.

Because increased activity of SGK1 correlates with renal epithelial Na⁺ channel activity which leads to hypertension through the increase of renal resorption of sodium (Lifton 1996; Staessen et al., 2003; Warnock 2001), it was conclusive that depending on the combination of allelic variants of SGK1 an increase in renal Na⁺-resorption may occur which in turn will increase the blood pressure (Busjahn et al. 2002).

### SUMMARY OF THE INVENTION

The expression of Connective tissue growth factor (CTGF) in fibroplasts is central for the induction of fibrosis related to a wide variety of diseases. The current invention unexpectedly demonstrates that the increased expression of CTGF strongly correlates with the presence and up-regulation of the serum and glucocorticoid inducible kinase SGK1.

In more detail the present invention discloses that SGK1 has two novel key functions (i) the signalling of mineralocorticoids to salt appetite and (ii) the mediation of mineralocorticoid induced formation of CTGF and cardiac fibrosis.

The data demonstrate for example of cardiac fibrosis that the SGK1 kinase plays a crucial role in fibrosing disease in general. Excessive transcription of SGK1 has been observed in diabetic nephropathy, glomerulonephritis, Crohn's disease, lung fibrosis, liver cirrhosis and fibrosing pancreatitis. The functional significance of excessive SGK1 transcription in cardiac fibrosis has been explored and the experienced in the art can readily expand the presented observations to other fibrosing diseases that have not yet been explored throughout this work, however strongly suggest that SGK1 actively participates in the pathophysiology of said diseases.

Generation of CTGF-in fibroblasts derived from SGK1 knockout-mice cannot be induced by deoxycorticosterone, an agent which is well known for the induction of fibrosis. On the other hand the hormone induces a pronounced expression of CTGF in fibroblasts derived from normal mice having fully functional SGK1. Thus SGK1 is a powerful regulator of CTGF driven fibrosis.

Because CTGF expressed in fibroblasts is the most important mediator for the induction of fibrosis, the inhibition of SGK1 allows interference with CTGF expression leading to suppression of fibrosis. SGK1 with this central role in the disease promoting process has therefore in addition to the natural inherent function some unexpected and new functions related to diseases leading to fibrosis:

The invention delivers as well a method for determining the predisposition, progression, regression or onset of a fibrosing disease and this is done by measuring the up-regulation or down-regulation of expression of SGK1 in tissue samples and specimens in conjunction with the status of the CTGF. Samples taken from diseased individuals may furthermore allow the analysis of selected SGK1 single nucleotide expression polymorph variants in such samples and their correlation with predisposition for disease.

Another aspect is related to screening methods for identifying new drug candidates that modulate disease related SGK1. Modulators especially useful according to this invention are compounds that interfere with SGK1 function thus preventing up-regulation of CTGF expression and activity. Inhibitors of SGK1 are especially useful to treat subjects suffering from symptoms of diseases selected from the group of “fibroproliferative disorders”: Fibrosis caused by irradiation, tumors, vascularization, granulomatous diseases, organ and graft rejection, lupus erythematosus, arteriosclerosis, hypoxia, oxidative stress, myocardial infarction and ischemia, cardiac hypertrophy and fibrosis, glomerulonephritis and glomerulosclerosis, renal fibrosis, diabetes mellitus, fibrosing pancreatitis, liver cirrhosis, steatohepatitis and biliary fibrosis, fibrosing and inflammatory bowel diseases, peptic ulcers, intra-abdominal adhesions, peritoneal fibrosing in peritoneal dialysis, pulmonary fibrosis, fibrosing alveolitis, pulmonary sarcoidosis and/or asthma, ovarian dysfunction, uterus myoma, arthritis, muscle pain/myalgia and fasciitis, scleroderma, keloid, gingival hypertrophy, scar formation, disturbing formation of scars or connective tissue in the cornea, occular fluid and in the retina, glaucoma, cerebral lesions including cerebral infarction, Alzheimer's disease, wound healing, healing after tooth extraction, bone healing and growth, post-fracture bone healing.

The drug screening approach performed according to this invention has led to the discovery of SGK1 directed therapeutic compounds. Two different classes of compounds, one belonging to the class of Acylhydrazone derivatives and the other belonging to Pyridopyrimidine derivatives have been identified. Selected SGK1 inhibiting compounds in pharmaceutical compositions comprising a pharmaceutically effective carrier, excipient or diluent are useful for the treatment of the various diseases leading to fibrosis. It is central to this invention that the screening methods used to identify new drugs with the desired therapeutic profile are not restricted to the compounds disclosed in this application. Moreover, it is evident to the expert that a one step approach or a two step approach for screening of SGK1 modulating compounds may be useful to apply. The first step of such a screening includes the identification of compounds that interfere with the SGK1 kinase activity. Various assay formats are available and a preferred assay uses the measurement of SGK1 catalyzed radioactive phosphorylation of a protein or peptide as substrate together with the .γATP. In the presence of an SGK1 inhibitory compound no or decreased radioactive signal is detectable. In a second readout system the SGK1 inhibiting compounds are monitored for their potential to interfere with CTGF expression and, however measuring other read-out activities may be useful as well. In addition or instead of measuring CTGF it may as well be considered to measure procollagen, intergrin α5 or proteoglycan.

### DETAILED DESCRIPTION OF THE INVENTION

To explore whether SGK1 may be involved in the signalling of cardiac fibrosis a pellet continuously releasing DOCA (2.4 mg/day) was implanted into both sgk1+/+ and sgk1−/− mice along with 1% NaCl in the drinking water.

Prior to treatment, blood pressure was similar in sgk1−/− and sgk1+/+ mice as were plasma Na+, Cl—, Ca2+ and phosphate concentrations, glomerular filtration rate, urinary flow rate and renal electrolyte elimination.

In both sgk1−/− and sgk1+/+ mice DOCA/high salt treatment for 18 days led to statistically significant increases in blood pressure and urinary output of NaCl and water. The effect was paralleled by significant increases in urinary output of Ca2+ and phosphate, typical sequalae of extracellular volume expansion 12, 13. DOCA induced a significant hypokalemia in sgk1+/+ but not in sgk1−/− mice, implicating a role for SGK1 in mineralocorticoid-regulated renal K+ excretion

The increase in blood pressure in sgk1−/− mice is consistent with significant upregulation of renal Na+ reabsorption and as shown here, this SGK1-independent upregulation of renal salt reabsorption by DOCA-mediated activation of mineralocorticoid receptors is, apparently, sufficient to induce net renal NaCl retention and thus to increase blood pressure.

The DOCA/high salt-induced increases in plasma Na+ concentration, urinary flow rate, absolute and fractional NaCl excretion were, however, significantly blunted in sgk1−/− compared to sgk1+/+ mice. In view of the defective stimulation of renal Na+ reabsorption in the sgk1−/− mice the opposite, i.e. enhanced rather than decreased urinary NaCl output in those mice was expected.

Thus a new finding was that SGK1 may contribute to mineralocorticoid-induced salt appetite. To verify this possibility, mice were placed in metabolic cages with access to two drinking bottles where bottle 1 always contained tap water. Switching bottle 2 from tap water to 1% NaCl did not significantly alter water intake from bottle 1 in sgk1+/+ mice and induced a pronounced increase in salt intake from bottle 2 only after implanting the DOCA pellet, consistent with DOCA-induced salt appetite (FIG. 1). This response, however, was significantly attenuated in sgk1−/− mice. A significant difference in fluid intake from bottle 2 persisted between sgk1+/+ and sgk1−/− mice even after offering tap water in bottle 2, indicating that sgk1+/+ mice maintained a greater search for salt than sgk1−/− mice. These data provide the first evidence that SGK1 plays a dual role in mineralocorticoid-regulated Na+ homeostasis involving not only inhibition of output by stimulation of renal Na+ reabsorption but also stimulation of uptake by mineralocorticoid induced salt appetite (6, 7). SGK1 may similarly participate in the regulation of salt appetite by glucocorticoids (2) and a SGK1-dependent increase of salt intake may contribute to the enhanced extracellular fluid volume and blood pressure during stress conditions (15). Moreover, enhanced salt appetite and subsequent increased salt uptake and extracellular fluid expansion may contribute to the higher blood pressure values of individuals carrying a common polymorphism within the SGK1 gene, affecting as many as 5% of unselected Caucasians (16).

Despite an identical increase in blood pressure, the effects of DOCA/high salt on kidney growth and proteinuria were significantly blunted in the sgk1−/− mouse. Moreover, heart morphology was dramatically different in sgk1−/− and sgk1+/+ mice. A 18 day DOCA/high salt treatment led to marked fibrosis of the heart in sgk1+/+ mice but remained without any appreciable effect on the heart in sgk1−/− mice (FIG. 2a). Quantitative analysis of the severity of fibrosis, which was assessed by measuring the area of fibrosis and then expressing it as a percentage of total area, revealed a statistically significant difference between the degree of fibrosis between DOCA/high salt treated sgk1+/+ and sgk1−/− mice (FIG. 2b). No significant fibrosis was observed in sgk1+/+ or sgk1−/− mice prior to DOCA/high salt treatment (FIG. 2b).

The enhanced cardiac fibrosis observed in the DOCA/high salt treated sgk1+/+ mice was paralleled by altered transcriptional regulation of several genes as determined by microarray analysis. In sgk1+/+ mice a 48 h DOCA/high salt treatment induced several genes involved in the pathogenesis of fibrosis, such as procollagens, integrin α5, proteoglycan 4 and connective tissue growth factor CTGF (FIG. 2c), paralleling the increase of SGK1 transcription (2.10±0.14 fold increase in SGK1/GAPDH copy number in DOCA/high salt treated sgk1+/+ mice compared to untreated sgk1+/+ mice as analysed by real-time PCR). In contrast none of these genes were induced by DOCA/high salt in sgk1−/− mice.

As the DOCA/high salt treated sgk1+/+ mice exhibited a greater uptake of salt compared to the sgk1−/− mice (FIG. 1) and prolonged enhanced salt intake has been previously reported to stimulate cardiac fibrosis in the absence of mineralocorticoids (17) we performed a further series of experiments where sgk1+/+ mice were offered DOCA plus 1% saline and sgk1−/− mice DOCA plus 2% saline in order to determine whether the differences in the degree of cardiac fibrosis between the sgk1+/+ and sgk1−/− mice were secondary to greater salt uptake by the sgk1+/+ mice. Even though under those conditions, the fluid uptake in sgk1−/− mice (n=5) was 2.2±0.4 fold higher and Na+ intake was thus more than 4-fold higher in sgk1−/− mice than in sgk1+/+ mice (n=5), cardiac CTGF expression (used in this instance as a marker of cardiac fibrosis), as analysed by Western blotting, was significantly increased only in cardiac tissue from sgk1+/+ mice (untreated: 0.9±0.2 vs DOCA/1% salt: 4.7±1.0, arbitrary units of CTGF/β-tubulin densitometric analysis, (P<0.01)) and not sgk1−/− mice (untreated: 1.5±1.0 vs DOCA/2% salt: 1.7±0.7, arbitrary units of CTGF/β-tubulin densitometric analysis).

Hypokalemia as described above may be cardiotoxic and thus lead to reparative fibrosis (18). Thus, the excessive hypokalemia in the sgk1+/+ DOCA/high salt treated mice could have contributed to the observed cardiac fibrosis. However, supplementation of rats undergoing mineralocorticoid/high salt treatment with KCl has been previously reported to have no effect on the degree of fibrosis and collagen content of the heart (19).

CTGF, a member of the CCN (ctgf/cyr61/nov) gene family (20), is known to be a key mediator of matrix protein formation (21,22), and upregulation of collagen and integrin a5 transcription can be secondary to CTGF expression (23,24). We therefore further explored the role of DOCA in the regulation of CTGF protein levels. According to Western blot analysis, treatment with DOCA/high salt (for 18 days) markedly upregulated CTGF expression in cardiac tissue from sgk1+/+ mice but was unaltered in sgk1−/− mice (FIGS. 3a,b). Similarly, stimulation of primary cultures of mouse lung fibroblasts isolated from sgk1+/+ and sgk1−/− mice showed elevated CTGF levels only in the sgk1+/+ cells after stimulation with DOCA (10 μM, 24 h) (FIGS. 3c,d). The requirement of SGK1 for the in vitro stimulation of CTGF expression in cultured fibroblasts suggests that the differences in mineralocorticoid-induced CTGF formation do not require differences in blood pressure, electrolyte metabolism or plasma concentrations between sgk1−/− and sgk1+/+ mice, even though those and further functional parameters may well contribute to altered cardiac function and fibrosis during mineralocorticoid excess. The SGK1-dependent upregulation of CTGF expression provides an explanation for the decisive role of this kinase in the signalling of enhanced matrix deposition. The observations do not, however, exclude additional signalling pathways involving SGK1 and leading to cardiac fibrosis.

### BRIEF DESCRIPTION OF THE DRAWINGS

FIG. 1: SGK1 is required for stimulation of NaCl uptake by DOCA.

Daily volume drunk from bottle 1 (tap water, left panel) or from bottle 2 (tap water or saline, right panel) by the SGK1 knockout mice (sgk1−/−, open symbols) or their wild type littermates (sgk1+/+, closed symbols) prior to and during DOCA treatment

FIG. 2: SGK1 is required for DOCA-induced development of cardiac fibrosis and for alterations in gene expression.

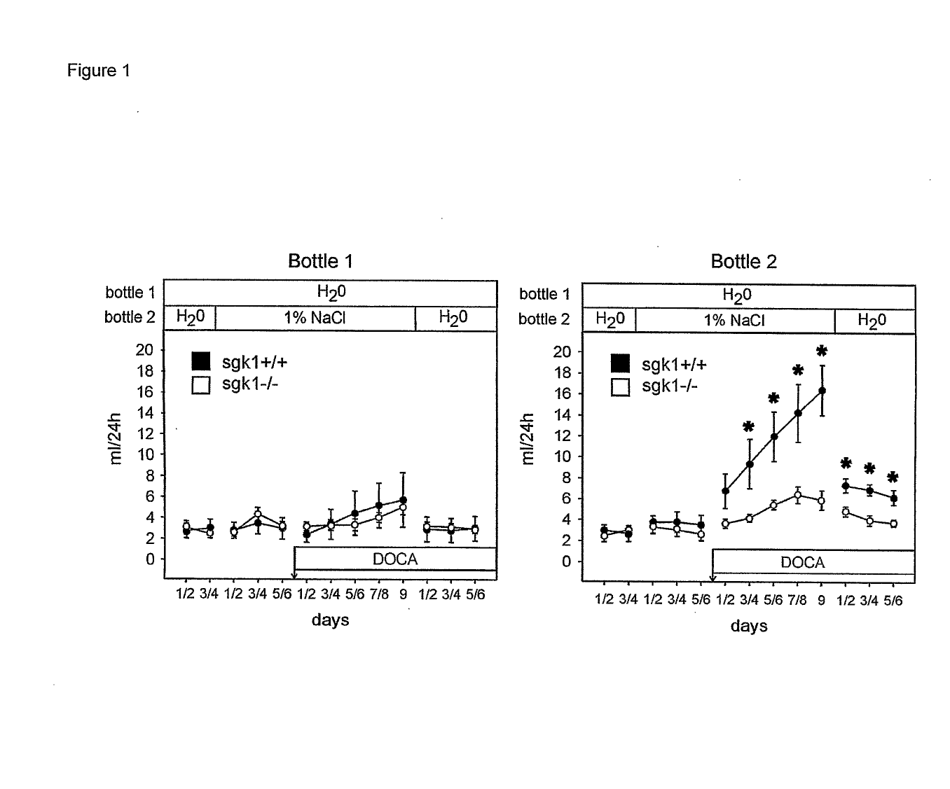

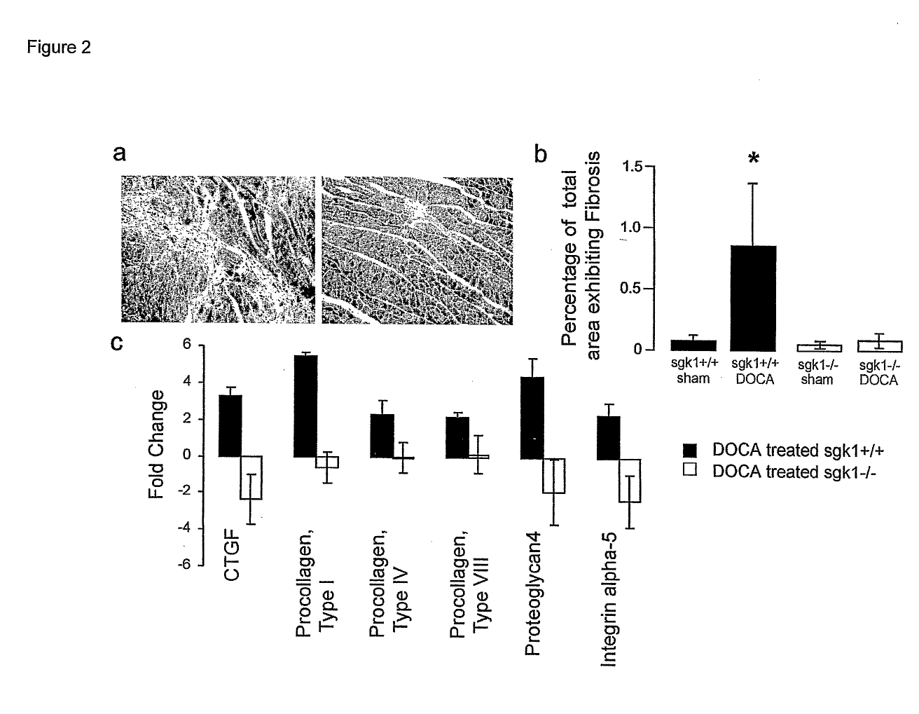

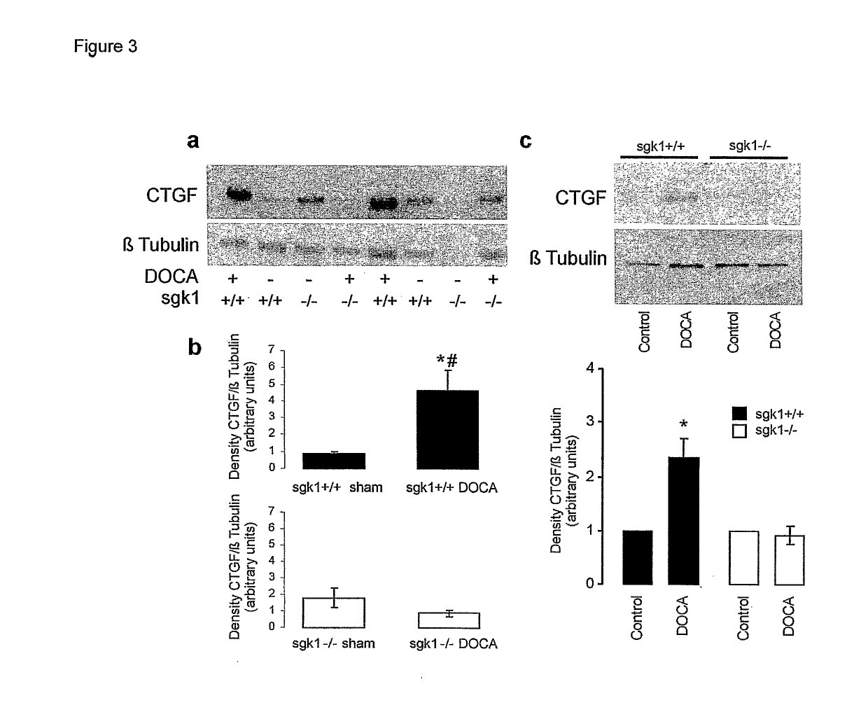

a: H&E and Masson's trichrome staining of cardiac tissue (×150 magnification) from wild type (sgk1+/+, left) and SGK1 knockout (sgk1−/−, right) mice following DOCA/high salt diet treatment for 18 days. b: Graph illustrating the degree of fibrosis (the fibrotic areas are presented as the percentage of total area) observed in DOCA/high salt treated sgk1+/+ and sgk1−/− (mean±SEM, n=6, * significant difference (P<0.05)). c: Microarray analysis showing arithmetic means±SEM (4 comparisons in 2 animals) of the fold change in connective tissue growth factor (CTGF), procollagen type I, IV, and VIII, proteoglycan 4 and integrin α5 transcript levels compared to untreated mice following a 2 day DOCA/high salt diet treatment of SGK1 knockout mice (sgk1−/−, open columns) and wild type littermates (sgk1+/+, closed columns).

FIG. 3: DOCA enhances CTGF expression in hearts and primary mouse lung fibroblast from sgk1+/+ but not sgk1−/− mice.

a: Representative Western blot of CTGF and β-tubulin levels in hearts from control and DOCA/high salt treated (18 days) SGK1 knockout mice (sgk1−/−) and wild type littermates (sgk1+/+). b: Densitometric analysis of the CTGF western blots expressed as a ratio of β-tubulin (mean±SEM, n=3-5, * significant difference between sgk1+/+ sham and sgk1+/+ DOCA, # significant difference between sgk1+/+ DOCA and sgk1−/− DOCA (P<0.05)) c: Representative Western blots of CTGF and β-tubulin expression in DOCA treated mouse lung fibroblasts and d: arithmetic means±SEM (lower panel, n=6, * Significant difference, P<0.05) of CTGF protein abundance (as a function of β-tubulin expression) in fibroblasts derived from SGK1 knockout animals (sgk1−/−, open columns) and wild type littermates (sgk1+/+, closed columns).

### ADDITIONAL METHODS AND MATERIALS

### Example 1

### Animal Experimentation

Mice deficient in SGK1 (sgk1−/−) were generated as previously described 9. Wild type (sgk1+/+) and SGK1 knockout (sgk1−/−) mice were implanted with a 21 day release 50 mg DOCA pellet (Innovative Research of America, Sarasota, Fla.) in the neck area (28) during anesthesia (intraperitoneal medetomidin 0.5 mg/kg+midazolam 5 mg/kg+fentanyl 0.05 mg/kg which was reversed by subcutaneous atipamezol 2.5 mg/kg+flumazenil 0.5 mg/kg+naloxon 1.2 mg/kg). One day before implantation of DOCA pellets, sgk1−/− and sgk1+/+ mice were weighed and placed individually in metabolic cages (Tecniplast Hohenpeissenberg, Germany) for basal 24 hour urine collection. Mice had free access to a standard mouse diet (Altromin, Heidenau, Germany) and tap water and/or 1% or 2% NaCl. The inner wall of the metabolic cages was siliconized and urine was collected under water-saturated oil. Systolic arterial blood pressure was determined by the tail-cuff method before and on days 1, 2, 4, 6, 10 and 14 of DOCA/1% NaCl treatment 29. On day 18 of DOCA/high salt treatment, 24 hour urine and body weight were again determined, animals anesthetized (intraperitoneal ketamine and xylazine) and 200 μl of blood withdrawn into heparinized capillaries by puncturing the retro-orbital plexus. Plasma and urinary concentrations of Na+ and K+ were measured by flame photometer (ELEX 6361, Eppendorf, Germany), Cl— concentrations by electrometric titration (Chloridometer 6610, Eppendorf, Germany), Ca2+, phosphorus and creatinine were assessed by commercial diagnostic kits (Sigma, Munich, Germany).

### Example 2

### Microscopy

Hearts from untreated or DOCA/high salt treated (18 days) sgk1+/+ and sgk1−/− mice were quickly removed under anesthesia, the weight determined and fixed in 4% paraformaldehyde/0.1 M sodium phosphate buffer (pH 7.2) overnight and embedded in paraffin. Dewaxed 5 μm thick heart muscle sections were stained with H&E and Masson's trichrome (30). Stained paraffin sections were analysed on a Zeiss Axioplan microscope (Zeiss, Jena, Germany). Areas were measured on digitized images using an Axiocam video camera (Zeiss, Jena, Germany) using the manufacture's software (Axiovision, Zeiss, Jena, Germany). Total tissue areas were measured with a 4× objective; fibrotic areas were identified and quantified using a 20× objective. The degree of fibrosis was then calculated as a percentage of total tissue area.

### Example 3

### Microarray Analysis

Total RNA was isolated from hearts obtained from untreated or DOCA/high salt (48 h) treated sgk1+/+ and sgk1−/− mice using the Qiagen RNeasy Fibrous Tissue Midi Kit according to the manufacture's instructions (Qiagen, Hilden, Germany). Using total RNA from hearts of DOCA/high salt or sham-treated sgk1−/− and sgk1+/+ mice, second-strand syntheses were generated using a commercially available kit (Invitrogen Life Technologies, Rockville, Md.) and an oligo d(T)24 T7 primer. cRNA was generated using biotin-labelled CTP and UTP by in vitro transcription using a T7 promoter-coupled double stranded cDNA as template and the T7 RNA transcript labelling kit (ENZO Diagnostics, Farmingdale, N.Y.). The cRNA was fragmented and hybridised to the mouse genome MOE430A oligonucleotide array chip (Affymetrix, Santa Clara, Calif.). The array chips were then stained using phycoerythrin conjugated streptavidin (Molecular Probes, Invitrogen Life Technologies, Rockville, Md.) and the fluorescence intensities were determined using a laser confocal scanner (Agilent, Affymetrix, Santa Clara, Calif.). The intensity of the scanned images was analysed using Microarray Suite Version 5 (Affymetrix, Santa Clara, Calif.). Global scaling was applied to all arrays such that the mean intensity of each array was equivalent. In global scaling, the raw signal value of each probe cell was multiplied by a scaling factor. Genes whose expression significantly varied with a signal log ratio of 0.5 were identified using Data Mining Tool (Affymetrix, Santa Clara, Calif.).

### Example 4

### SGK1 Modulating Compounds

4.1. Compounds of the General Formula I and Pharmaceutical Useful Derivates, Salts, Solutions and Stereoisomeres thereof Including Mixtures. 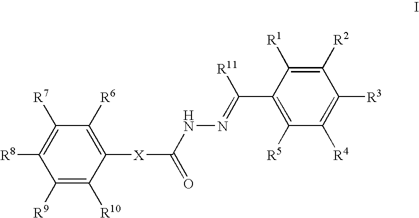
wherein
R¹, R⁵ is either H, OH, OA, OAc or Methyl,
R², R, R⁴, R⁶, R⁷, R⁸, R⁹, R¹⁰ is either
H, OH, OA, OAc, OCF₃, Hal, NO₂, CF₃, A, CN, OSO₂CH₃, SO₂CH₃, NH₂ or COOH,
   R¹¹ H or CH₃,
   A Alkyl with 1, 2, 3 or 4 C-atoms,
   X CH₂, CH₂CH₂, OCH₂ or —CH(OH)—,
   Hal F, Cl, Br or I
   Compound According to Formula I Selected from the Following Group of Compounds:
(3-Hydroxy-phenyl)-acidic acid-(4-hydroxy-2-methoxy-benzyliden)-hydrazid,
(3-Hydroxy-phenyl)-acidic acid-[1-(4-hydroxy-2-methoxy-phenyl)-ethyliden]-hydrazid,
(3-Methoxy-phenyl)-acidic acid-(4-hydroxy-2-methoxy-benzyliden)-hydrazid.
Phenylacidic acid-(3-fluor-4-hydroxy-benzyliden)-hydrazid,
(4-Hydroxy-phenyl)-acidic acid-(4-hydroxy-2-methoxy-benzyliden)-hydrazid,
(3,4-Dichlor-phenyl)-acidic acid-(4-hydroxy-2-methoxy-benzyliden)-hydrazid,
m-Tolyl-acidic acid-(4-hydroxy-2-methoxy-benzyliden)-hydrazid,
o-Tolyl-acidic acid-(4-hydroxy-2-methoxy-benzyliden)-hydrazid,
(2-Chlor-phenyl)-acidic acid-(4-hydroxy-2-methoxy-benzyliden)-hydrazid,
(3-Chlor-phenyl)-acidic acid-(4-hydroxy-2-methoxy-benzyliden)-hydrazid,
(4-Fluor-phenyl)-acidic acid-(4-hydroxy-2-methoxy-benzyliden)-hydrazid,
(2-Chlor-4-fluor-phenyl)-acidic acid-(4-hydroxy-2-methoxy-benzyliden)-hydrazid,
(3-Fluor-phenyl)-acidic acid-(4-hydroxy-2-methoxy-benzyliden)-hydrazid,
(3-Methoxy-phenyl)-acidic acid-(4-hydroxy-benzyliden)-hydrazid, (3-Methoxy-phenyl)-acidic acid-(4-hydroxy-2,6-dimethyl-benzyliden)-hydrazid,
(3-Methoxy-phenyl)-acidic acid-(3-fluor-4-hydroxy-benzyliden)-hydrazid, (3-Methoxy-phenyl)-acidic acid-[1-(4-hydroxy-2-methoxy-phenyl)-ethyliden]-hydrazid,
(3-Methylsulfonyloxy-phenyl)-acidic acid-(4-hydroxy-2-methoxy-benzyliden)-hydrazid,
(3,5-Dihydroxy-phenyl)-acidic acid-(4-hydroxy-2-methoxy-benzyliden)-hydrazid,
(3-Fluor-phenyl)-acidic acid-(3-fluor-4-hydroxy-benzyliden)-hydrazid,
(3-Methoxy-phenyl)-acidic acid-(4-acetoxy-2-methoxy-benzyliden)-hydrazid,
(3-Trifluormethyl-phenyl)-acidic acid-(4-hydroxy-2-methoxy-benzyliden)-hydrazid,
3-(3-Methoxy-phenyl)-propionsäure-(4-hydroxy-2-methoxy-benzyliden)-hydrazid,
(3-Methoxy-phenyl)-acidic a cid-(2,4-dihydroxy-benzyliden)-hydrazid,
(3-Methoxy-phenoxy)-acidic acid-(4-hydroxy-2-methoxy-benzyliden)-hydrazid,
(3-Nitro-phenyl)-acidic acid-(4-hydroxy-2-methoxy-benzyliden)-hydrazid,
(3-Methoxy-phenyl)-acidic acid-(5-chlor-2-hydroxy-benzyliden)-hydrazid,
(3-Methoxy-phenyl)-acidic a cid-(2-hydroxy-5-nitro-benzyliden)-hydrazid,
2-Hydroxy-2-phenyl-acidic acid-(4-hydroxy-2-methoxy-benzyliden)-hydrazid,
(3-Methoxy-phenyl)-acidic acid-(2-ethoxy-4-hydroxy-benzyliden)-hydrazid,
(3-Brom-phenyl)-acidic acid-(4-hydroxy-2-methoxy-benzyliden)-hydrazid,
(3-Methoxy-phenyl)-acidic acid-[1-(4-hydroxy-phenyl)-ethyliden]-hydrazid,
(3,5-Difluor-phenyl)-acidic acid-(4-hydroxy-2-methoxy-benzyliden)-hydrazid,
(3-Hydroxy-phenyl)-acidic acid-(4-hydroxy-2-methyl-benzyliden)-hydrazid,
(3-Hydroxy-phenyl)-acidic acid-(2-ethoxy-4-hydroxy-benzyliden)-hydrazid,
(3-Hydroxy-phenyl)-acidic acid-(2-methoxy-4-hydroxy-6-methyl-benzyliden)-hydrazid,
(2-Fluor-phenyl)-acidic acid-(2-methoxy-4-hydroxy-benzyliden)-hydrazid
   4.2. Compounds of the General Formula II and Pharmaceutical Useful Derivates, Salts, Solutions and Stereoisomeres thereof Including Mixtures. 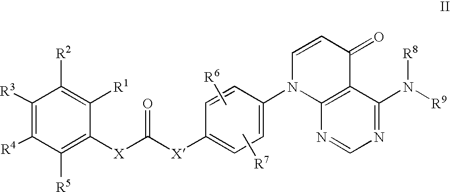
   wherein
   R¹, R², R³,
   R⁴, R⁵ is either H, A, OH, OA, Alkenyl, Alkinyl, NO₂, NH₂, NHA, NA₂, Hal, CN, COOH, COOA,
   —OHet, —O-Alkylen-Het, —O-Alkylen-NR⁸R⁹ or CONR⁸R⁹,
   two groups selected from R¹, R², R³, R⁴, R⁵ or as well —O—CH₂—CH₂—, —O—CH₂—O— or —O—CH₂—CH₂—O—,
      R⁶, R⁷ is either H, A, Hal, OH, OA or CN,
      R⁸, R⁹ is either H or A,
      Het
      Is a saturated or unsaturated heterocycle with 1 to 4 N-, O- and/or S-atoms, substituted by one or several Hal, A, OA, COOA, CN or Carbonyloxigen (═O)
      A Alkyl with 1 to 10 C-atoms, wherein 1-7H-atoms may be replaced by F and/or Chlorine,
      X, X′ is either NH or is missing
      Hal F, Cl, Br or I
      Compound According to Formula II Selected from the Following Group of Compounds:
1-[4-(4-Amino-5-oxo-5H-pyrido[2,3-d]pyrimidin-8-yl)-phenyl]-3-(2-fluor-5-trifluormethyl-phenyl)-urea,
1-[4-(4-Amino-5-oxo-5H-pyrido[2,3-d]pyrimidin-8-yl)-phenyl]-3-(4-chlor-5-trifluormethyl-phenyl)-urea,
1-[4-(4-Amino-5-oxo-5H-pyrido[2,3-d]pyrimidin-8-yl)-phenyl]-3-(2,4-difluorphenyl)-urea,
1-[4-(4-Amino-5-oxo-5H-pyrido[2,3-d]pyrimidin-8-yl)-phenyl]-3-(2,6-difluor-phenyl)-urea,
1-[4-(4-Amino-5-oxo-5H-pyrido[2,3-d]pyrimidin-8-yl)-phenyl]-3-(3-fluor-5-trifluormethyl-phenyl)-urea,
1-[4-(4-Amino-5-oxo-5H-pyrido[2,3-d]pyrimidin-8-yl)-phenyl]-3-(4-fluor-5-trifluormethyl-phenyl)-urea,
1-[4-(4-Amino-5-oxo-5H-pyrido[2,3-d]pyrimidin-8-yl)-phenyl]-3-(4-methyl-5-trifluormethyl-phenyl)-urea,
1-[4-(4-Amino-5-oxo-5H-pyrido[2,3-d]pyrimidin-8-yl)-phenyl]-3-(2,3,4,5,6-pentafluor-phenyl)-urea,
1-[4-(4-Amino-5-oxo-5H-pyrido[2,3-d]pyrimidin-8-yl)-phenyl]-3-(2,4-dibrom-6-fluor-phenyl)-urea,
1-[4-(4-Amino-5-oxo-5H-pyrido[2,3-d]pyrimidin-8-yl)-phenyl]-3-(2-fluor-6-trifluormethyl-phenyl)-urea,
1-[4-(4-Amino-5-oxo-5H-pyrido[2,3-d]pyrimidin-8-yl)-phenyl]-3-(2-fluor-5-methyl-phenyl)-urea,
1-[4-(4-Amino-5-oxo-5H-pyrido[2,3-d]pyrimidin-8-yl)-phenyl]-3-(2,3,4-trifluor-phenyl)-urea,
1-[4-(4-Amino-5-oxo-5H-pyrido[2,3-d]pyrimidin-8-yl)-phenyl]-3-(4-brom-2,6-difluor-phenyl)-urea,
1-[4-(4-Amino-5-oxo-5H-pyrido[2,3-d]pyrimidin-8-yl)-phenyl]-3-(2-fluor-3-trifluormethyl-phenyl)-urea,
1-[4-(4-Amino-5-oxo-5H-pyrido[2,3-d]pyrimidin-8-yl)-phenyl]-3-[2-(1-tert.-butyloxycarbonyl-piperidin-4-yl)-phenyl]-urea,
N-[4-(4-Amino-5-oxo-5H-pyrido[2,3-d]pyrimidin-8-yl)-phenyl]-2,4-dichlor-benzamid,
N-[4-(4-Amino-5-oxo-5H-pyrido[2,3-d]pyrimidin-8-yl)-phenyl]-4-chlor-5-trifluormethyl-benzamid,
N-[4-(4-Amino-5-oxo-5H-pyrido[2,3-d]pyrimidin-8-yl)-phenyl]-2-fluor-5-trifluormethyl-benzamid,
1-[4-(4-Amino-5-oxo-5H-pyrido[2,3-d]pyrimidin-8-yl)-phenyl]-3-[3-chlor-5-trifluormethyl-2-(piperidin-4-yloxy)-phenyl]-urea,
1-[4-(4-Amino-5-oxo-5H-pyrido[2,3-d]pyrimidin-8-yl)-phenyl]-3-[(2-fluor-5-(2-dimethylamino-ethoxy)-phenyl]-urea,
1-[4-(4-Amino-5-oxo-5H-pyrido[2,3-d]pyrimidin-8-yl)-phenyl]-3-[5-fluor-2-(piperidin-4-yloxy)-phenyl]-urea,
1-[4-(4-Amino-5-oxo-5H-pyrido[2,3-d]pyrimidin-8-yl)-phenyl]-3-[4-chlor-5-trifluormethyl-2-(piperidin-4-yloxy)-phenyl]-urea,
1-[4-(4-Amino-5-oxo-5H-pyrido[2,3-d]pyrimidin-8-yl)-phenyl]-3-[2-(piperidin-4-yloxy)-phenyl]-urea,
1-[4-(4-Amino-5-oxo-5H-pyrido[2,3-d]pyrimidin-8-yl)-phenyl]-3-[2-fluor-5-(2-diethylamino-ethoxy)-phenyl]-urea,
1-[4-(4-Amino-5-oxo-5H-pyrido[2,3-d]pyrimidin-8-yl)-phenyl]-3-[2-fluor-5-[2-(piperidin-1-yl)-ethoxy]-phenyl]-urea,
1-[4-(4-Amino-5-oxo-5H-pyrido[2,3-d]pyrimidin-8-yl)-phenyl]-3-[4-fluor-2-(2-dimethylamino-ethoxy)-phenyl]-urea,
1-[4-(4-Amino-5-oxo-5H-pyrido[2,3-c]pyrimidin-8-yl)-phenyl]-3-[4-fluor-2-(2-diethylamino-ethoxy)-phenyl]-urea,
1-[4-(4-Amino-5-oxo-5H-pyrido[2,3-d]pyrimidin-8-yl)-phenyl]-3-[3-chlor-4-[2-(morpholin-4-yl)-ethoxy]-phenyl]-urea,
1-[4-(4-Amino-5-oxo-5H-pyrido[2,3-d]pyrimidin-8-yl)-phenyl]-3-[4-fluor-2-[2-(morpholin-4-yl)-ethoxy]-phenyl]-urea,
1-[4-(4-Amino-5-oxo-5H-pyrido[2,3-d]pyrimidin-8-yl)-phenyl]-3-[3-chlor-4-(2-dimethylamino-ethoxy)-phenyl]-urea,
1-[4-(4-Amino-5-oxo-5H-pyrido[2,3-d]pyrimidin-8-yl)-phenyl]-3-[3-chlor-4-(2-diethylamino-ethoxy)-phenyl]-urea,
1-[4-(4-Amino-5-oxo-5H-pyrido[2,3-d]pyrimidin-8-yl)-phenyl]-3-[4-chlor-2-(2-dimethylamino-ethoxy)-phenyl]-urea,
1-[4-(4-Amino-5-oxo-5H-pyrido[2,3-d]pyrimidin-8-yl)-phenyl]-3-[2-chlor-5-(2-diethylamino-ethoxy)-phenyl]-urea,

### Example 5

### SGK1 Nucleotide Polymorphism

The nucleotide sequence defining intron 6 of facultative hypertensive patients is . . . aattacattCgcaacccag . . . , whereas the nucleotide sequence representing a healthy population is . . . aattacattTgcaacccag . . . . The sequences are available through accession number GI 2463200 Position 2071. The exon 8 sequences of facultative hypertensive patients are either homozygotic . . . tactgaCttcggact . . . or . . . tactgaTttcggact . . . or heterozygotic . . . tactgaCttcggact . . . and . . . tactgaTttcggact . . . The sequences are available through accession number NM_{—}005627.2, Position 777.

A homozygotic individual with a TT nucleotide combination is protected even if simultaneously a CC single nucleotide polymorphism is presented in intron 6.

### Example 6

### Cell Culture

To harvest lung fibroblasts from sgk1+/+ and sgk1−/− mice (8-14 weeks old), whole lungs were removed and transferred to 90 mm cell culture dishes containing 2 ml of DMEM supplemented with 10% fetal calf serum, 100 U/ml Penicillin, 100 mg/ml Streptomycin and 2 mM L-glutamine (Gibco-Invitrogen, Karlsruhe, Germany). The tissue was cut into small pieces and cultured under standard cell culture conditions (37° C., 5% CO2). Cell growth was observed 2-4 days after initial plating. Fibroblasts were identified by positive staining for fibronectin and used in experiments between passages 2-6. Increase of SGK1 mRNA by DOCA treatment and absence of SGK1 in sgk1−/− mice lung fibroblasts was confirmed by realtime PCR (data not shown).

### Example 7

### Western Blot Analysis

Whole hearts from untreated and DOCA/high salt treated (18 days) sgk1+/+ and sgk1−/− mice were removed and immediately frozen in liquid nitrogen, the tissue was then homogenised using a glass homogeniser in lysis buffer containing 50 mM Tris-HCl, pH 7.4, 100 mM NaCl, 1 mM EDTA, 1 mM EGTA, 50 mM sodium fluoride, 5 mM sodium pyrophosphate, 1 mM sodium orthovanadate, 1% Triton X-100, 1% sodium deoxycholate, 1% sodium doecyl sulphate and protease cocktail inhibitor (Roche, Basel, Switzerland), the homogenates were centrifuged at 10,000 rpm, 4° C. for 15 min, the supernatant was removed and used for Western blotting. sgk1+/+ and sgk1−/− mouse lung fibroblasts in 60 mm culture dishes were serum deprived for 18 h prior to the addition of DOCA (10 μM), 24 h later the cells were lysed. Whole cell lysates (50 μg) and heart homogenates (70 μg) were separated by SDS-page (10% Tris-Glycine), transferred to nitrocellulose membranes, blocked for 1 h in blocking buffer (5% fat-free milk in PBS containing 0.1% Tween) and incubated overnight at 4° C. with a goat polyclonal CTGF primary antibody (diluted 1:400 in blocking buffer, Santa Cruz, Heidelberg, Germany). After incubation with a HRP-conjugated anti-goat secondary antibody (Santa Cruz, Heidelberg, Germany) visualization with ECL was performed according to manufacture's instructions (Amersham, Freiburg, Germany). Membranes were also probed with a primary β-tubulin (Santa Cruz, Heidelberg, Germany) antibody as a loading control. Densitometric analysis of CTGF was performed using Scion Image (Scion, Md., USA) and normalized using β-tubulin.

### REFERENCES

1. Radiotherapy/irradiation [Flanders et al., 2003; Gervaz et al., 2003; Quan et al., 2002; Vozenin-Brotons et al., 2003]
2. Tumors [Hishikawa et al., 1999; Kasaragod et al., 2001; Koliopanos et al., 2002; Li and Sarkar 2002; Moritani et al., 2003; Shimo et al., 2001] Vascularization
3. Granulomatous diseases [Babic et al., 1999; Shimo et al., 1999] [Inkinen et al., 2003]
4. Organ/graft rejection [Franceschini et al., 2003; Inkinen et al., 2001]
5. Lupus erythematosis [Bao et al., 2003]
6. Arteriosclerosis and Hypoxia [Harlow and Hillier 2002; Ruperez et al., 2003; Schober et al., 2002; Schober et al., 2003] [Honda et al., 2001; Kondo et al., 2002; Shimo et al., 2001]
7. Oxidative stress [Park et al., 2001]
8. Myocardial infarction and ischemia [Ohnishi et al., 1998; Simkhovich et al., 2003; Way et al., 2002]
9. Cardiac hypertrophy and fibrosis [Buchhorn et al., 2003; Chen et al., 2000; Finckenberg et al., 2001]
10. Glomerulonephritis and glomerulosclerosis [Ito et al., 2001; Kanemoto et al., 2003], [Chen et al., 2003; Gupta et al., 2000]
11. (Tubulo-interstitial) renal fibrosis [Gupta et al., 2000; Inoue et al., 2003]
12. Diabetes mellitus [Gilbert et al., 2003; Tikellis et al., 2004; Wada et al., 2002]
13. (Fibrosing) pancreatitis [di Mola et al., 1999; Schuppan et al., 2000; Vogelmann et al., 2001]
14. Liver cirrhosis, steatohepatitis and biliary fibrosis [Abou-Shady et al., 2000; Hayashi et al., 2002; Kamada et al., 2003; Kurikawa et al., 2003; Schuppan et al., 2000], [Paradis et al., 2001], [Sedlaczek et al., 2001]
15. Fibrosing and inflammatory bowel diseases [Dammeier et al., 1998; Schuppan et al., 2000]
16. Treated peptic ulcers [Lempinen et al., 2002]
17. Intra-abdominal adhesions [Thaler et al., 2002]
18. Peritoneal fibrosing in peritoneal dialysis [Zarrinkalam et al., 2003]
19. Pulmonary fibrosis [Allen et al., 1999; Atamas and White 2003; Bonniaud et al., 2003; Howell et al., 2001; Kelly et al., 2003]
20. Fibrosing alveolitis, pulmonary sarcoidosis and/or asthma [Millar 2000], [Allen et al., 1999], [Burgess et al., 2003]
21. Ovarian dysfunction [Harlow et al., 2002; Harlow and Hillier 2002]
22. Uterus myoma [Sampath et al., 2001]
23. Arthritis [Varga and Kahari 1997],
24. Muscle pain (myalgia) and fasciitis (Varga and Kahari 1997)
25. (Pseudo-)sclerodenna [Atamas and White 2003; Igarashi et al., 1996; Leask et al., 2004; Querfeld et al., 2000]
26. Treated keloid [Igarashi et al., 1996; Liu et al., 2003]
27. Gingival hypertrophy [Hong et al., 1999; Uzel et al., 2001]
28. Scar formation [Liu et al., 2003; Wada et al., 2002]
29. Disturbing formation of scars or connective tissue in the cornea [Ivarsen et al., 2003; Razzaque et al., 2003; van Setten et al., 2003], occular fluid [van Setten et al., 2002] and in the retina [Honda et al., 2001]
30. Glaucoma [Esson et al., 2004]
31. Cerebral lesions [Hertel et al., 2000] including cerebral infarction [Schwab et al., 2000]
32. Alzheimer's disease [Ueberham et al., 2003]
33. Wound healing [Flanders et al., 2003; Thomas and Harding 2002; Wang et al., 2001]
34. Post-fracture bone healing [Fukunaga et al., 2003; Kanyama et al., 2003; Nakanishi et al., 2001; Nakata et al., 2002; Pereira et al., 2000; Takigawa et al., 2003].

1. A method for altering Connective tissue growth factor (CTGF) activity and expression comprising, contacting cells expressing SGK1, SGK2, SGK3 with a substance that modulates glucocorticoid inducible kinases.

2. Use of the method according to claim 1 for the preparation of a medicament for the treatment of a fibroproliferative disorders caused by CTGF up- or down-regulation.

3. The method according to claim 2, wherein the disease is selected from the group of fibroproliferative disorders: Disease caused by irradiation, tumors, vascularization, granulomatous diseases, organ and graft rejection, lupus erythematosus, arteriosclerosis, hypoxia, oxidative stress, myocardial infarction and ischemia, cardiac hypertrophy and fibrosis, glomerulonephritis and glomerulosclerosis, renal fibrosis, diabetes mellitus, fibrosing pancreatitis, liver cirrhosis, steatohepatitis and biliary fibrosis, fibrosing and inflammatory bowel diseases, peptic ulcers, intra-abdominal adhesions, peritoneal fibrosis in peritoneal dialysis, pulmonary fibrosis, fibrosing alveolitis, pulmonary sarcoidosis and/or asthma, ovarian dysfunction, uterus myoma, arthritis, muscle pain/myalgia and fasciitis, scleroderma, keloid, gingival hypertrophy, formation of scars or connective tissue in diverse organs including in the cornea, occular fluid and in the retina, glaucoma, cerebral lesions including cerebral infarction, Alzheimer's disease, wound healing, healing after tooth extraction, bone healing and growth, bone fracture repair

4. A method for determining the progression, regression or onset of a fibroproliferative disorder by measuring the up-regulated expression of SGK1, SGK2 or SGK3 in tissue samples and specimens.

5. A method according to claim 4, wherein the SGK1 comprises a selected single nucleotide polymorph variant.

6. A method according to claim 1 for the diagnosis of disease, wherein the disease is selected from the group of: Disease caused by irradiation, tumors, vascularization, granulomatous diseases, organ and graft rejection, lupus erythematosus, arteriosclerosis, hypoxia, oxidative stress, myocardial infarction and ischemia, cardiac hypertrophy and fibrosis, glomerulonephritis and glomerulosclerosis, renal fibrosis, diabetes mellitus, fibrosing pancreatitis, liver cirrhosis, steatohepatitis and biliary fibrosis, fibrosing and inflammatory bowel diseases, peptic ulcers, intra-abdominal adhesions, peritoneal fibrosis in peritoneal dialysis, pulmonary fibrosis, fibrosing alveolitis, pulmonary sarcoidosis and/or asthma, ovarian dysfunction, uterus myoma, arthritis, muscle pain/myalgia and fasciitis, scleroderma, keloid, gingival hypertrophy, formation of scars or connective tissue in diverse organs including in the cornea, occular fluid and in the retina, glaucoma, cerebral lesions including cerebral infarction, Alzheimer's disease, wound healing, healing after tooth extraction, bone healing and growth, bone fracture repair.

7. Use of SGK1 inhibitors selected from the listed compounds having the general formula I or II for the manufacture of a medicament for the treatment of disorders caused by dysregulated Connective tissue growth factor.
